(19) United States
(12) Patent Application Publication
(10) Pub. No.: US 2026/0114812 A1
(43) Pub. Date: Apr. 30, 2026

(54) Microcatheter for Rapid Measurement of Deformable Material Mechanical Properties

(71) Applicant: Queen's University at Kingston, Kingston (CA)

(72) Inventors: Xian Wang, Kingston (CA); Zhaokai Wang, Kingston (CA)

(21) Appl. No.: 19/358,055

(22) Filed: Oct. 14, 2025

Related U.S. Application Data
(60) Provisional application No. 63/706,786, filed on Oct. 14, 2024.

(51) Int. Cl. A61B 5/00; A61L 29/02; A61L 29/06; A61L 29/14; A61M 25/01

(52) U.S. Cl. CPC A61B 5/6852; A61L 29/02; A61L 29/06; A61L 29/14; A61M 25/0158; A61B 2562/0219

(57) ABSTRACT

An instrument for measuring a mechanical property of a deformable tissue includes a rigid hollow tube housing a flexible magnetic microcatheter in a sliding fit. A length of the flexible magnetic microcatheter may be advanced from a distal end of the rigid hollow tube to engage the deformable material, and exhibits a deflection in response to an applied magnetic field. A relation of force and deflection of the flexible magnetic microcatheter engaged with the deformable material in response to the applied magnetic field is indicative of the mechanical property of the deformable material. The instrument may be used to measure deformable material mechanical properties in fields such as engineering and material science, and in medicine wherein the deformable material is biological tissue.

### RELATED APPLICATION

This application claims the benefit of the filing date of Application No. 63/706,786, filed on Oct. 14, 2024, the contents of which are incorporated herein by reference in their entirety.

### FIELD

The invention relates broadly to the field of measuring mechanical properties of materials. More specifically, the invention relates to apparatus and methods for measuring mechanical properties of deformable materials.

### BACKGROUND

The ability to measure mechanical properties of deformable materials is important in fields such as engineering and medicine. For example, understanding and characterizing how material properties change in response to an applied load or stress is critical to the design of structures and systems to prevent failure.

Tissue mechanical property measurement is becoming an important cancer diagnostic tool [1]. Currently, the mechanical properties of cancer tissues can be characterized by different techniques such as tensile and compression tests, microindentation, and atomic force microscopy (AFM) [2-4]. Despite extensive data on the mechanical properties that current measurement systems offer, current techniques suffer from either specific sample preparation or sophisticated measurement setup and procedure, which limits their application in assisting intraoperative histopathology [5]. In addition, most systems are limited to surface measurement or small region measurement, limiting the comprehensiveness of assessments inside the tissue.

### SUMMARY

According to one aspect of the invention there is provided an instrument, comprising: a rigid hollow tube; a flexible magnetic microcatheter housed within the rigid hollow tube in a sliding fit; wherein a length of the flexible magnetic microcatheter is adapted to be advanced from a distal end of the rigid hollow tube; wherein the length of the flexible magnetic microcatheter advanced from the distal end of the rigid hollow tube exhibits a deflection in response to an applied magnetic field.

In one embodiment the length of the flexible magnetic microcatheter advanced from the distal end of the rigid hollow tube is adapted to engage a deformable material and to deflect when engaged with the deformable material in response to the applied magnetic field.

In one embodiment a relation of force and deflection of the flexible magnetic microcatheter engaged with the deformable material in response to the applied magnetic field is indicative of a mechanical property of the deformable material.

In one embodiment the distal end of the rigid hollow tube is configured with a tip that is adapted to pierce a deformable material.

In one embodiment the distal end of the rigid hollow tube is configured as a needle.

In one embodiment the deformable material comprises biological tissue.

In one embodiment the flexible magnetic microcatheter comprises a polymer with embedded magnetic features.

In one embodiment the magnetic features comprise magnetic microparticles.

In one embodiment the magnetic features comprise NdFeB particles.

In one embodiment the polymer comprises polydimethylsiloxane (PDMS).

According to another aspect of the invention there is provided a method for measuring a mechanical property of a deformable material, comprising: engaging a flexible magnetic microcatheter with the deformable material; applying a magnetic field to the flexible magnetic microcatheter; wherein the magnetic field causes the flexible magnetic microcatheter to deflect and deform the deformable material; and determining the mechanical property of the deformable material according to a relation of force and deflection of the flexible magnetic microcatheter.

In one embodiment the method comprises housing the flexible magnetic microcatheter in a rigid hollow tube in a sliding fit; wherein a length of the flexible magnetic microcatheter is adapted to be advanced outwardly from and retracted inwardly to a distal end of the rigid hollow tube.

In one embodiment the method comprises piercing the deformable material with the distal end of the rigid hollow tube; advancing the length of the flexible magnetic microcatheter outwardly from the distal end of the rigid hollow tube to engage the deformable material; retracting the rigid hollow tube from the deformable material while the length flexible magnetic microcatheter is engaged with deformable material; and applying the magnetic field to the flexible magnetic microcatheter engaged with the deformable material.

According to another aspect of the invention there is provided apparatus for measuring a mechanical property of a deformable material, comprising: an instrument as described herein; a carriage adapted to control movement of the instrument towards and away from the deformable material and to enable engagement of the flexible magnetic microcatheter with the deformable material; a magnet adapted for movement with at least 2 degrees of freedom proximal to the length of the flexible magnetic microcatheter advanced from the distal end of the rigid hollow tube.

### BRIEF DESCRIPTION OF THE DRAWINGS

For a greater understanding of the invention, and to show more clearly how it may be carried into effect, embodiments will be described, by way of example, with reference to the accompanying drawings, wherein:

FIGS. 1A-1F are diagrams of a method for preparing a magnetic microcatheter, according to one embodiment.

FIGS. 2A and 2B are diagrams of a magnetic microcatheter mechanical measurement system according to one embodiment, wherein FIG. 2B shows steps for preforming a measurement using the embodiment.

FIG. 3 presents a series of photomicrographs of magnetic microcatheter deflection in air (upper row) and in agar (lower row) for different magnetic field strengths, according to one embodiment.

FIGS. 4A and 4B are plots of magnetic microcatheter deflection vs distance from magnet (FIG. 4A) and magnetic field strength (FIG. 4B), used to calibrate one embodiment.

FIGS. 5A and 5B are plots of deflection and force measurement, respectively, in air and agar, for a magnetic microcatheter according to one embodiment.

### DETAILED DESCRIPTION OF EMBODIMENTS

Described herein is a microcatheter fabricated from magnetic materials that may be controlled by an external applied magnetic field to perform mechanical measurements of deformable materials. Embodiments may be configured as a system for measuring one or more mechanical properties of biological tissue, or other deformable materials, and accordingly are adaptable for use in a wide range of applications such as, but not limited to, medicine, engineering, material science, and quality control. Embodiments may be easy to set up and may provide rapid measurements, e.g., requiring only several seconds for a tissue mechanical measurement. Embodiments have broad utility for measuring mechanical properties of deformable materials, and may be particularly useful in medical applications with fewer resources, such as in clinics, and in surgery rooms where there is a need for rapid mechanical tests.

In some embodiments the microcatheter may comprise a flexible polymer matrix body with embedded magnetic microparticles that can be externally actuated by applying a magnetic field and used for mechanical measurement of a deformable material. The microcatheter may be housed within a rigid hollow tube, also referred to herein as a needle or hollow needle, in a sliding fit that allows the microcatheter to be advanced outwardly from and retracted inwardly to the distal end of the hollow tube. The hollow tube may be implemented with a suitable rigid material which may be a metal such as, e.g., stainless steel, surgical grade steel, etc. Actuation, e.g., deflection, of a portion of the microcatheter that is advanced outwardly from the distal end of the hollow tube may be achieved using the applied magnetic field. Advancing and retracting the microcatheter from the distal end of the hollow tube may be achieved manually or using an actuator such as, e.g., a stepper motor, to the drive the microcatheter from the proximal end of the hollow tube. To facilitate this, the microcatheter may be attached to a stiff wire or similar feature that fits within the hollow tube and allows the microcatheter to be advanced from and retracted into the distal end of the hollow tube from the proximal end of the hollow tube. Fine control of the advancing and retracting of the microcatheter (i.e., longitudinal movement) may be achieved by, for example, providing a threaded adjuster at the proximal end of the hollow tube that provides a selected amount of longitudinal movement according to the pitch of the thread.

In one embodiment the flexible polymer matrix of the microcatheter body may be constructed from a resin of a polymer that is flexible when cured, such as, for example, silicone (e.g., polydimethylsiloxane (PDMS)) or, e.g., a copolymer which may be prepared from two or more polymers such as a copolymer of silicone with another polymer. Ther polymer resin may be mixed with magnetic particles (e.g., iron-bearing particles) such as, for example, NdFeB microparticles (e.g., around 5 micrometers in size). It will be appreciated that copolymerization may be used to enhance or tune physical properties of the microcatheter, e.g., through the cross-linking of polymer chains by adding molecular weight to the chain. In some cases, adding a soft polymer can reduce the modulus of a particularly rigid material (often a silicone-based material) to achieve a flexibility appropriate for a given application, type of deformable material being tested, etc. Also, for some polymers such as PDMS, elasticity of the cured polymer may be modified based on the amounts (i.e., ratio) of pre-polymer and curing agent used. For example, whereas a ratio of 10:1 is common, the ratio may be optimized to increase or decrease the elastic modulus while avoiding the presence of adhesive properties that can occur.

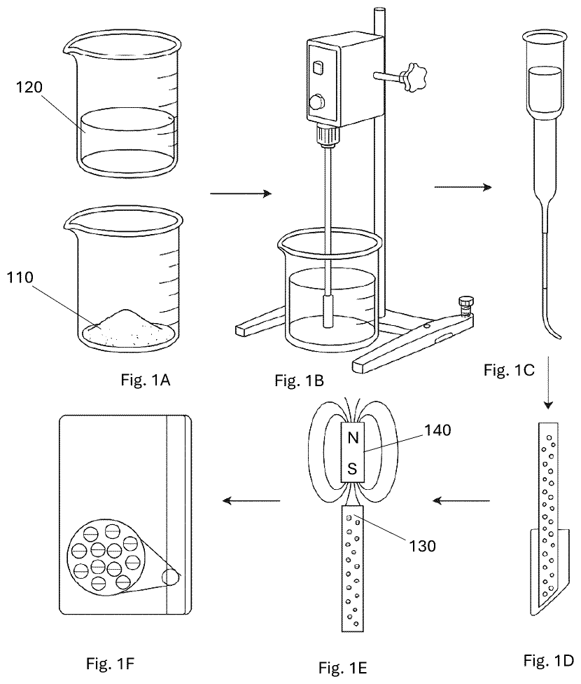

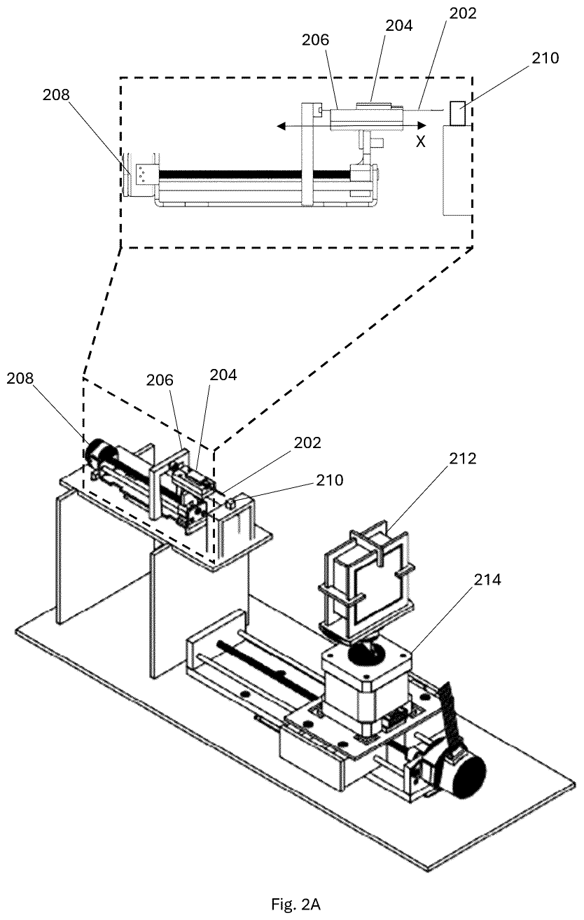

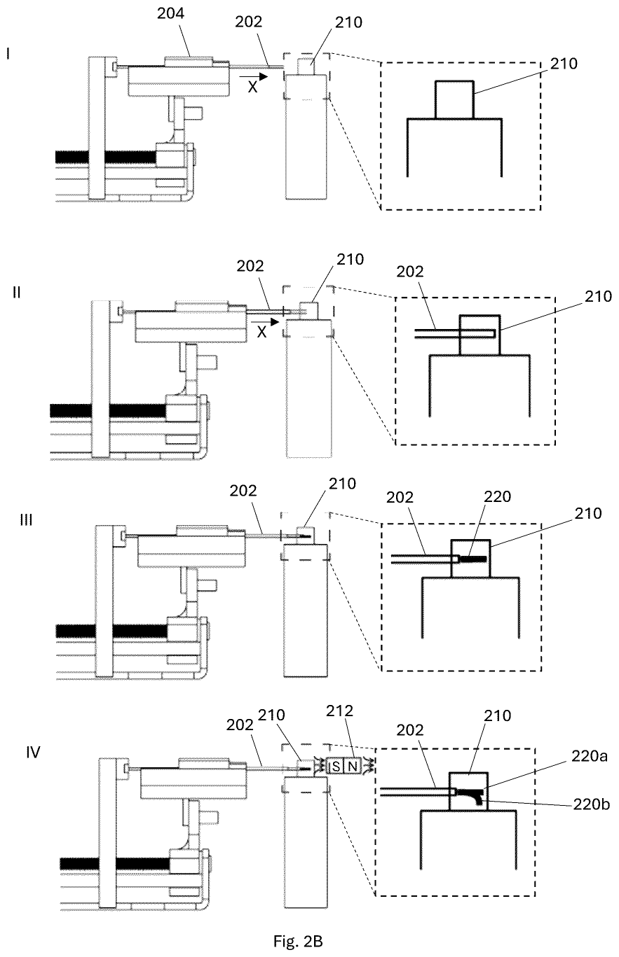

In one embodiment, shown in FIGS. 1A-1F, NdFeB microparticles 110 (about 20 volume %) were added into uncured PDMS resin (Sylgard 184, Dow Corning) 120 (FIG. 1A) and mixed using an overhead electrical stirrer for 10 minutes (FIG. 1B). About 10 wt % of curing agent containing platinum catalyst was added in a subsequent mixing for 45 s under the same conditions. The resin was then injected into a microtube (i.e., a mold) with an inner diameter of 640 μm and subsequently cured (FIG. 1C). After curing and demolding (FIG. 1D), the tip of the microcatheter 130 was magnetized using a permanent magnet 140 (FIG. 1E) such that the NdFeB microparticles became magnetized (inset, FIG. 1F) and then the microcatheter was assembled onto the end of a steel wire. In some embodiments, the steel wire may extend beyond the proximal end of the hollow tube in which the microcatheter is housed and used to advance and retract the microcatheter in the distal end of the hollow tube. In this example the microcatheter had a length of about 6 mm and a diameter of about 640 μm. In other embodiments the length of the microcatheter be, e.g., 2 mm-15 mm, according to what may be appropriate for a given application. The diameter may be sized according to the inner diameter of the hollow tube or needle used, e.g., 0.3 mm to 0.8 mm as a practical range for gauge 24 to gauge 28 needles.

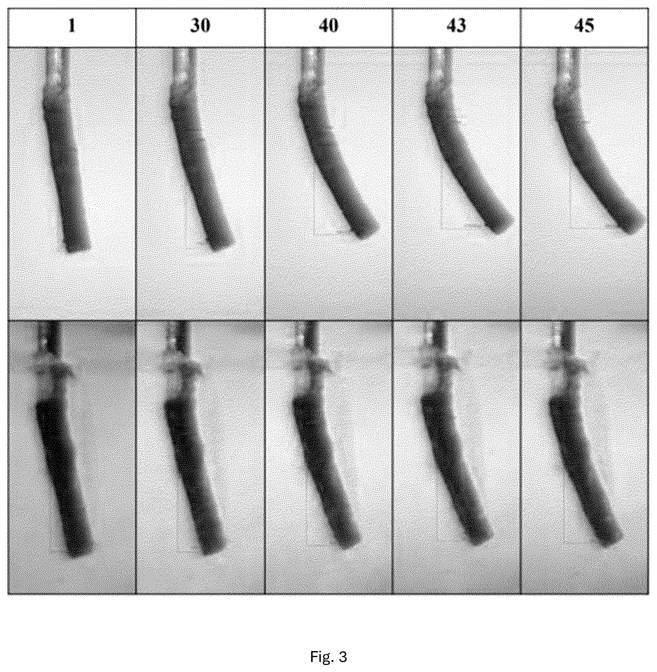

Actuation of the microcatheter may be achieved by disposing a strong magnet (e.g., a permanent magnet or an electromagnet) in close proximity to the portion of the microcatheter that is advanced outwardly from the distal end of the hollow tube and controlling the magnet's position and/or orientation in relation to the microcatheter. For example, actuation may be achieved by controlling the magnet in 2-degrees-of-freedom (DOF). A set-up used for preliminary testing and measurements of an embodiment is shown in FIG. 2A, and its operation is shown in FIG. 2B, steps (i) to (iv). Preliminary testing was performed with mouse brain tissue samples which were obtained through the Animal Care Facility of Queen's University at Kingston (Kingston, ON, Canda) in accordance with institutional and national guidelines for the care and use of laboratory animals. However, as noted above, embodiments may be adapted for measurements of any deformable material.

Referring to FIG. 2A in which the dashed lines show a close-up view of the apparatus used to move the hollow needle housing the microcatheter, the hollow needle 202 was secured in a needle holder 204 mounted to a carriage assembly 206 that was driven by a stepper motor 208 along the longitudinal X axis (i.e., toward and away from a tissue sample 210 and a magnet 212) as indicated by arrows X. The magnet 212 was mounted to an apparatus 214 that allowed adjustment of the magnet position in 2 DOF. Referring to FIG. 2B in which the dotted lines show close-up views of the tissue and hollow needle/magnetic catheter at each step I to IV to conduct a measurement, the hollow needle 202 was driven by the stepper motor toward the tissue 210 as shown by arrow X at step I until the distal end of the hollow needle 202 punctured and entered the tissue 210, as shown at step II. At step III the magnetic microcatheter 220 inside the hollow needle 202 was extended into the tissue 210 by pushing the proximal end of the steel wire to which it was attached, and fixed in the desired position to engage the tissue 210 and the hollow needle 2 was retracted substantially out of the tissue sample while a portion of the magnetic microcatheter 220 extended from the distal end of the hollow needle 202 remained inside the tissue 210. For the measurement, the magnet 212 was moved along the X-axis to gradually change the magnetic field applied to the microcatheter 220. By controlling the motion of the magnet, the resulting changing magnetic field drives the microcatheter to deflect, e.g., from 220a to 220b as shown at step IV of FIG. 2B where the small curved arrows represent the magnetic field, and cause tissue deformation with a known force, achieving the tissue mechanical measurement by relation of force and deformation δ. By comparing deflections of the microcatheter in air and in tissue samples the deflection force exerted on the catheter may be calculated, thereby enabling measurement of the elastic property of the tissue sample. In some embodiments an electromagnet may be used, which allows the magnetic force of the magnetic to be controlled by varying the amount of electrical current applied to it.

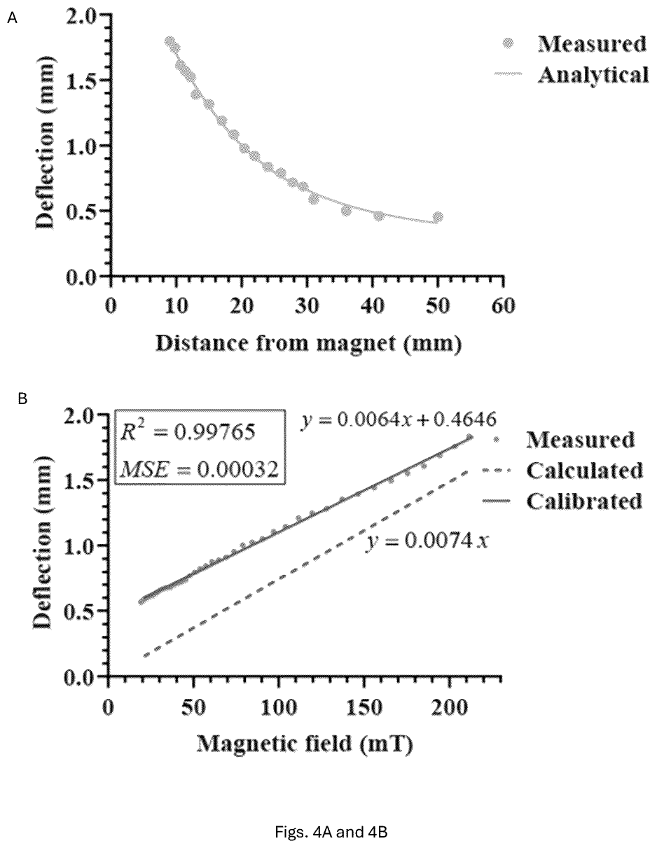

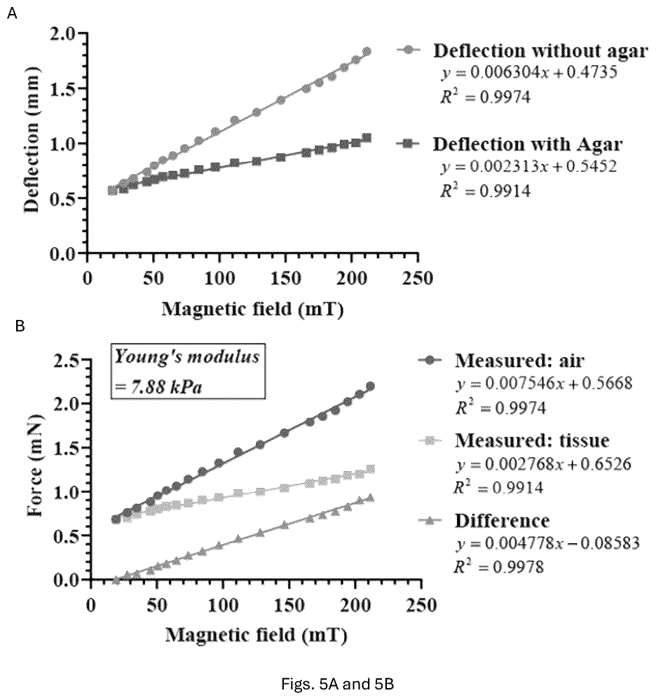

Calibration experiments were conducted with microcatheter deflection measured by a microscope. FIG. 3 shows photographs of microcatheter deflection for different magnetic field strengths (i.e., magnet position) in air (top row) and agar (bottom row). By controlling magnet position using a step model, the relationship between the catheter deflection and the magnetic force was quantified (FIG. 4A). By fitting two coefficients, the measured results and calculated results correlate very well with R²=0.99765 (FIG. 4B). By comparing the deflection error in air and in an agar block (FIG. 5A), the drag force exerted by the agar sample was determined (i.e., the difference in deflection in air and agar in FIG. 3). With a force of 0.94 mN and a deformation of 1.06 mm, Young's modulus of the agar gel was quantified to be 7.88 kPa (FIG. 5B).

From the above examples it is readily apparent that embodiments may be configured for measurement in vitro or in vivo of tissue mechanical properties in medical and life science applications. As non-limiting examples, for quantifying the mechanical properties of lung nodules, breast tissue, brain tissue, and their relationship to cancer, as well as other biological materials such as fibrin gel and collagen. It is also readily apparent that embodiments may be adapted for measuring deformable biological and non-biological materials in a wide range of applications such as, but not limited to, research, engineering, reliability testing, and quality control.

### Incorporation by Reference

The contents of all cited publications are incorporated herein by reference in their entirety.

### Equivalents

While the invention has been described with respect to illustrative embodiments thereof, it will be understood that various changes may be made to the embodiments without departing from the scope of the invention. Accordingly, the described embodiments are to be considered exemplary and the invention is not to be limited thereby.

### REFERENCES

1. Massey, A., et al., Nat. Rev. Phys., 2024, 6, 269.
2. Harris, A. R. et al., PNAS, 2012, 109, 16449.
3. Menichetti, A., et al., Int. J. Eng. Sci., 2020, 155, 103355.
4. Efremov, et al., Soft matter, 2020, 16, 64.
5. Navindaran, K., et al., J. Mech. Behav. Biomed. Mater., 2023, 138, 105575.

1. An instrument, comprising:
a rigid hollow tube;
a flexible magnetic microcatheter housed within the rigid hollow tube in a sliding fit;
wherein a length of the flexible magnetic microcatheter is adapted to be advanced from a distal end of the rigid hollow tube;
wherein the length of the flexible magnetic microcatheter advanced from the distal end of the rigid hollow tube exhibits a deflection in response to an applied magnetic field.

2. The instrument of claim 1, wherein the length of the flexible magnetic microcatheter advanced from the distal end of the rigid hollow tube is adapted to engage a deformable material and to deflect when engaged with the deformable material in response to the applied magnetic field.

3. The instrument of claim 2, wherein a relation of force and deflection of the flexible magnetic microcatheter engaged with the deformable material in response to the applied magnetic field is indicative of a mechanical property of the deformable material.

4. The instrument of claim 1, wherein the distal end of the rigid hollow tube is configured with a tip that is adapted to pierce a deformable material.

5. The instrument of claim 1, wherein the distal end of the rigid hollow tube is configured as a needle.

6. The instrument of claim 2, wherein the deformable material comprises biological tissue.

7. The instrument of claim 1, wherein the flexible magnetic microcatheter comprises a polymer with embedded magnetic features.

8. The instrument of claim 7, wherein the magnetic features comprise magnetic microparticles.

9. The instrument of claim 7, wherein the magnetic features comprise NdFeB particles.

10. The instrument of claim 7, wherein the polymer comprises polydimethylsiloxane (PDMS).

11. A method for measuring a mechanical property of a deformable material, comprising:
engaging a flexible magnetic microcatheter with the deformable material;
applying a magnetic field to the flexible magnetic microcatheter;
wherein the magnetic field causes the flexible magnetic microcatheter to deflect and deform the deformable material; and
determining the mechanical property of the deformable material according to a relation of force and deflection of the flexible magnetic microcatheter.

12. The method of claim 11, comprising housing the flexible magnetic microcatheter in a rigid hollow tube in a sliding fit;
wherein a length of the flexible magnetic microcatheter is adapted to be advanced outwardly from and retracted inwardly to a distal end of the rigid hollow tube.

13. The method of claim 12, comprising piercing the deformable material with the distal end of the rigid hollow tube;
advancing the length of the flexible magnetic microcatheter outwardly from the distal end of the rigid hollow tube to engage the deformable material;
retracting the rigid hollow tube from the deformable material while the length flexible magnetic microcatheter is engaged with deformable material; and
applying the magnetic field to the flexible magnetic microcatheter engaged with the deformable material.

14. The method of claim 11, wherein the deformable material comprises biological tissue.

15. The method of claim 11, wherein the flexible magnetic microcatheter comprises a polymer with embedded magnetic features.

16. The method of claim 15, wherein the magnetic features comprise magnetic microparticles.

17. The method of claim 15, wherein the magnetic features comprise NdFeB particles.

18. The method of claim 15, wherein the polymer comprises polydimethylsiloxane (PDMS).

19. Apparatus for measuring a mechanical property of a deformable material, comprising:
the instrument of claim 1;
a carriage adapted to control movement of the instrument towards and away from the deformable material and to enable engagement of the flexible magnetic microcatheter with the deformable material;
a magnet adapted for movement with at least 2 degrees of freedom proximal to the length of the flexible magnetic microcatheter advanced from the distal end of the rigid hollow tube.

20. The apparatus of claim 19, wherein the deformable material is biological tissue.
